(19) United States
(12) Patent Application Publication
(10) Pub. No.: US 2026/0026863 A1
(43) Pub. Date: Jan. 29, 2026

(54) ELECTROSURGICAL INSTRUMENT AND METHOD OF DETECTING TISSUE ACCUMULATION ON END EFFECTOR

(71) Applicant: Cilag GmbH International, Zug (CH)

(72) Inventors: John E. Brady, Liberty Township, OH (US); Curtis A. Maples, Cincinnati, OH (US); Kip M. Rupp, New Richmond, OH (US); Meredith N. Lusk, Dearborn Heights, MI (US); Chad P. Boudreaux, Cincinnati, OH (US); Madison K. Vanosdoll, Cincinnati, OH (US); Steven M. Boronyak, Cincinnati, OH (US); David J. Maher, Philadelphia, PA (US); Raymond E. Parfett, Cincinnati, OH (US); Cameron D. McLain, Deer Park, OH (US); Daniel V. Boguszewski, Cincinnati, OH (US)

(21) Appl. No.: 18/783,696

(22) Filed: Jul. 25, 2024

(51) Int. Cl. A61B 18/14; A61B 18/00

(52) U.S. Cl. CPC A61B 18/1445; A61B 2018/00619; A61B 2018/0063; A61B 2018/00678; A61B 2018/00773; A61B 2018/00875; A61B 2018/0097; A61B 2018/1455; A61B 2018/1467

(57) ABSTRACT

A surgical instrument includes an end effector and a controller. The end effector includes a first jaw, a second jaw, and an electrode associated with the first jaw. The first jaw and the second jaw can move between an open position and a clamped position in order to grasp tissue. Further, the electrode emits a therapeutic energy cycle to seal the grasped tissue. The controller can measure an impedance parameter associated with the end effector and compare the measured impedance parameter with an upper predetermined threshold. Further the controller can generate a cleaning instruction or alter an energy delivery algorithm of the RF therapeutic energy cycle if the measured impedance parameter is greater than the upper predetermined threshold.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio.

Buildup of surgical debris on the end-effector of an advanced bipolar device can result in worse sticking performance, premature termination of energy delivery algorithms, and shunting energy away from the tissue to be sealed onto other tissue on the jaw. These results pose a risk to seal quality. Accumulation of surgical debris on a jaw may also limit the capability of sensing technologies implemented in the jaw, such as bioimpedance spectroscopy, or optical sensing modalities, as the sensor measurements will be influences by material built up on the electrodes in addition to the target tissue to seal. A surgeon will use their own experience and judgement of when buildup of surgical debris on jaws has reached a threshold that requires cleaning of the jaws.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts an illustrative impedance triangle;

FIG. 7 depicts a set of illustrative example waveforms;

FIG. 8 depicts another set of illustrative example waveforms;

FIG. 9 depicts another illustrative example waveform;

FIG. 10 depicts another illustrative example waveform;

FIG. 11 depicts another set of illustrative example waveforms;

FIG. 12 depicts another illustrative example waveform;

FIG. 13 depicts another illustrative example waveform;

FIG. 14A depicts a cross-sectional view of the end effector of FIG. 2, wherein the jaws are in the open position prior to initial use;

FIG. 14B depicts a cross-sectional view of the end effector of FIG. 2, wherein the jaws are in the open position after a plurality of activations;

FIG. 15 depicts a graph of impedance measurements of an end effector in the open position over the course a plurality of activations;

FIG. 16 depicts a flowchart of an illustrative method utilizing subtherapeutic impedance sensing while jaws of an end effector are in an open position to determine if the end effector needs to be cleaned;

FIG. 17A depicts an elevational side view of an alterative end effector, wherein a pair of jaws of the end effector are in a closed position with eschar;

FIG. 17B depicts an elevational side view of the end effector of FIG. 17A, wherein the jaws are in an open position with eschar;

FIG. 18 depicts a flowchart of an illustrative method utilizing the opening rate of change of jaws of an end effector to determine if the end effector needs to be cleaned;

FIG. 19 depicts a cross-sectional view of the end effector of FIG. 17A, with the pair of jaws in closed position with no eschar;

FIG. 20 depicts a cross-sectional view of the end effector of FIG. 17A, with the pair of jaws in closed position with eschar;

FIG. 21 depicts a flowchart of an illustrative method of utilizing a measured jaw gap in the clamped position to determine if the end effector needs to be cleaned;

FIG. 22 depicts a scatter plot of minimum impedance over a series of activations during illustrative use;

FIG. 23 depicts a flowchart of an illustrative method of utilizing an impedance value during a therapeutic energy activation cycle to determine if the end effector needs to be cleaned; and

FIG. 24 depicts a flowchart of an illustrative method of utilizing a mean impedance value of multiple therapeutic energy activation cycles to determine if the end effector needs to be cleaned.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

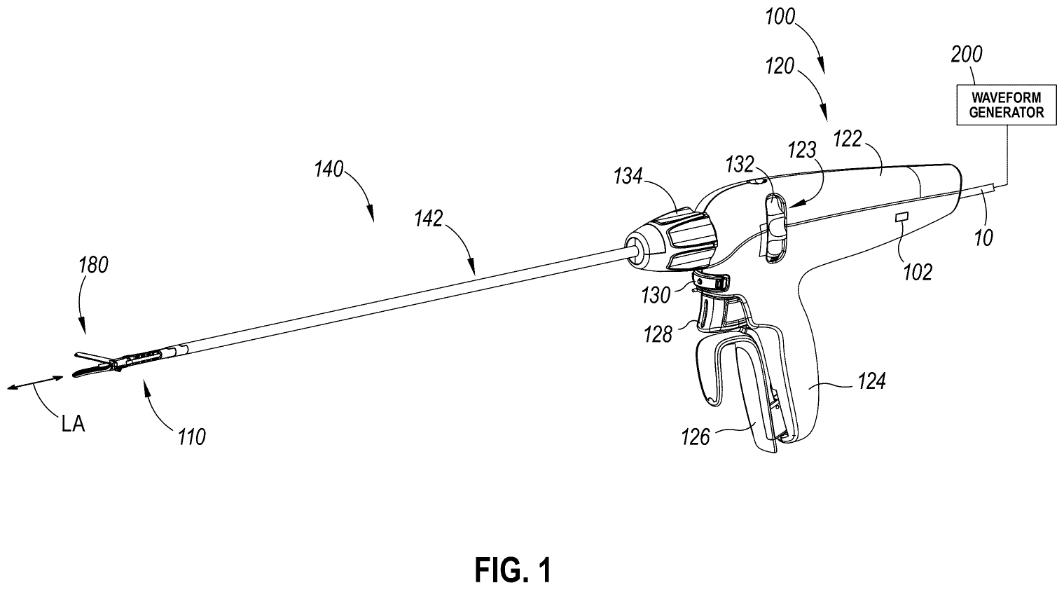

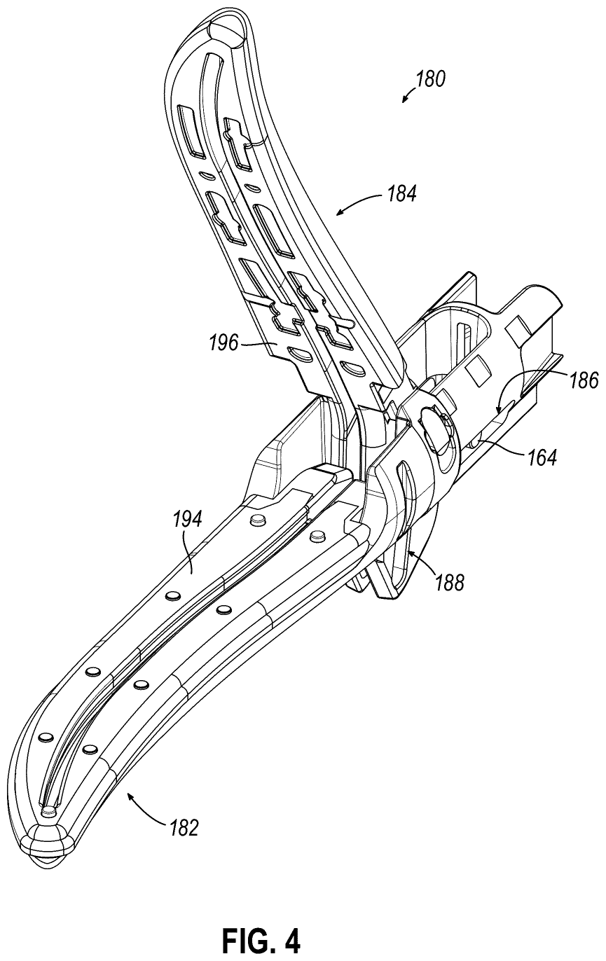

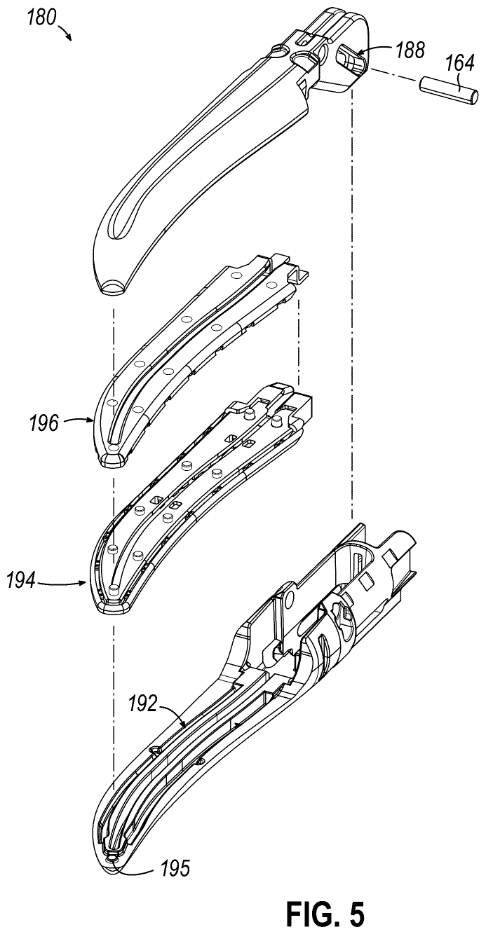

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. Application of RF energy may be applied to electrodes (194, 196) utilizing a suitable RF therapeutic energy activation cycle/algorithm. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

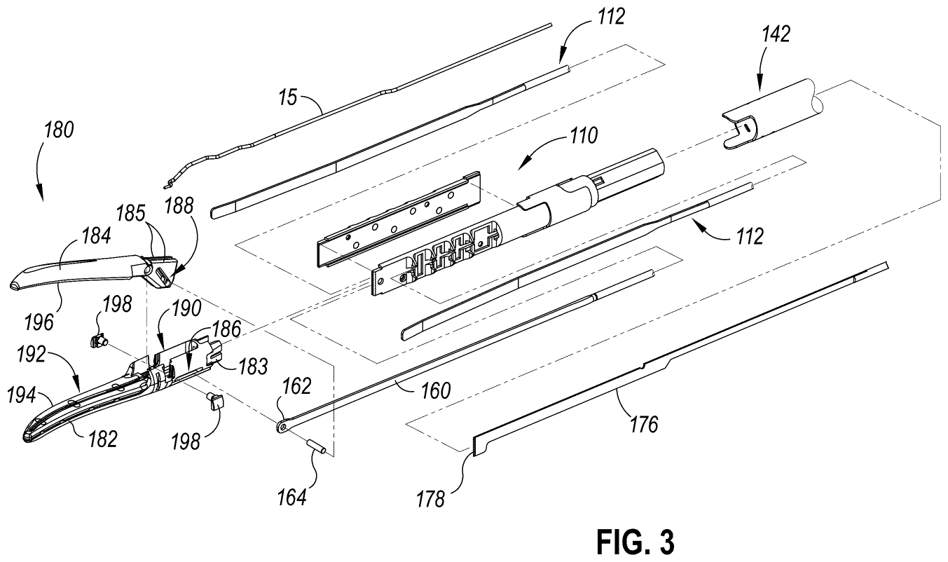

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

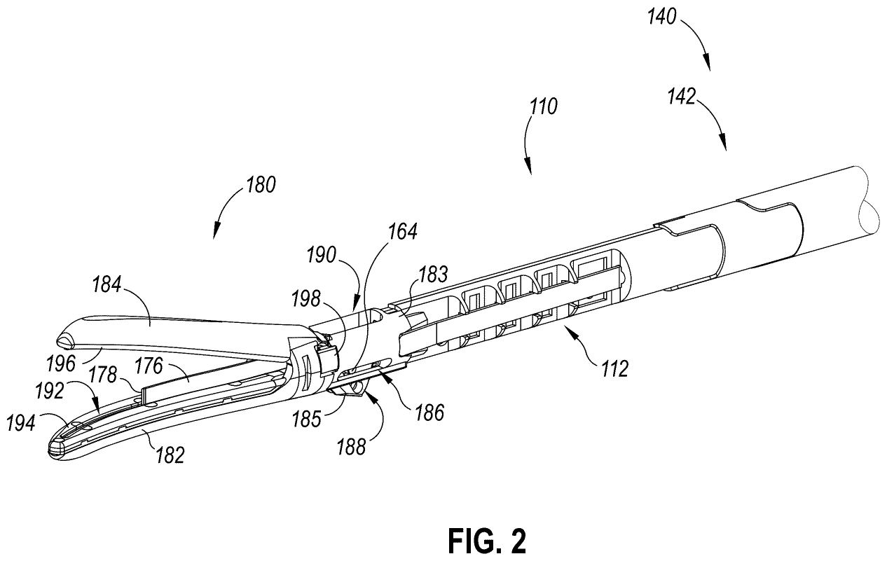

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Sensing Tissue Impedance for Determinations of Tissue State

Electrosurgical instrument (100) discussed above is configured to clamp tissue using end effector (180). Once securely clamped, electrodes (194, 196) in end effector (180) apply a non-therapeutic (i.e., low voltage) waveform to the tissue; and sensor devices measure the returning waveform to calculate and measure the impedance of the tissue. In one example, one or more electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine a phase and a magnitude of the impedance of the tissue within jaws (182, 184). A processor may then relay information associated with a state of the tissue, such as, for example, tissue type, tissue phase, tissue margin, and the like. Using this associated information, a system inclusive of such electrosurgical instrument (100) cannot only verify that the proper tissue is clamped between jaws (182, 184), but can also determine if any non-tissue material is present between jaws (182, 184), and/or if a proper seal has been created after applying the therapeutic RF energy.

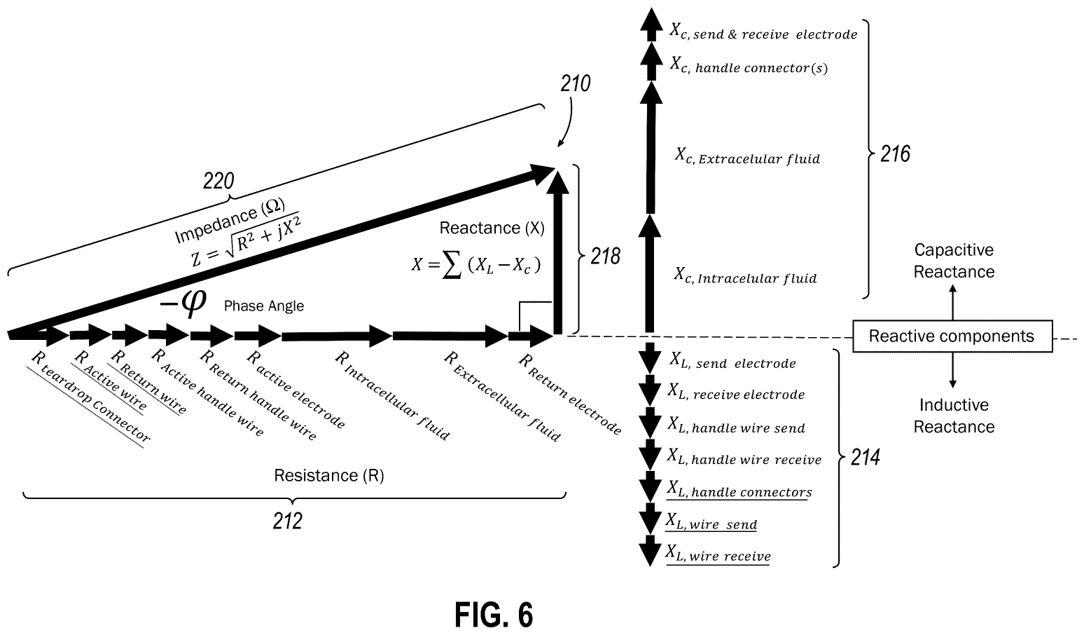

FIG. 6 shows an illustrative impedance triangle (210). As would be understood by one skilled in the art, human tissues may tend to be capacitive in nature, while wires, tool, staples, implants, etc. may tend to be inductive in nature. Thus, as can be seen by the illustrative impedance triangle (210), the “resistance” of each object in the circuit is measured (212) using the waveform and sensor electrodes (194, 196). Such system can also determine the “capacitive reactance” of each object in the circuit and the inductive reactance of each object in the circuit. For example, the send and receive electrodes (194, 196), the send and receive handle wires, the handle connector and the send and receive wires (e.g., included in power cable 10 (see FIG. 1)) all have inductive reactance (214). Additionally, the send and receive electrodes (194, 196), the handle connector, the extracellular fluid, and the intracellular fluid all have capacitive reactance (216). The “reactance” (218) can then be calculated by determining the difference between the capacitive reactance and the inductive reactance using:

$$X = \sum ( {X}_{L} - {X}_{C} ) . \qquad Equation 1$$

As shown in FIG. 6, the “impedance” 1406 can then be determined using:

$$Z = \sqrt{{R}^{2} + j {X}^{2}} \qquad Equation 2$$

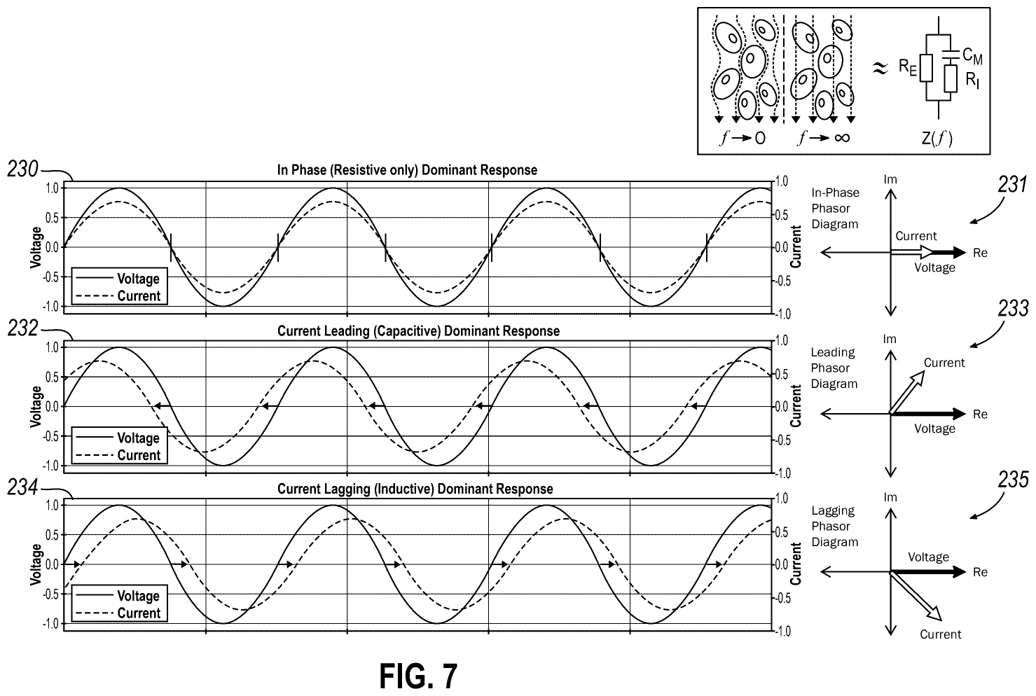

FIG. 7 shows a set of illustrative example waveforms. As would be understood by one skilled in the art, if a circuit only contains resistive items, the current and voltage will remain in phase such as shown in a first graph (230) and a first phasor diagram (231). Alternatively, if the circuit has capacitive objects, or more capacitive than inductive, the voltage wave will lead the current wave such as shown in a second graph (232) and a second phasor diagram (233). Finally, if the circuit has inductive objects, or more inductive objects than capacitive objects, the voltage will lag behind the current, such as shown in a third graph (234) and third phasor diagram (235).

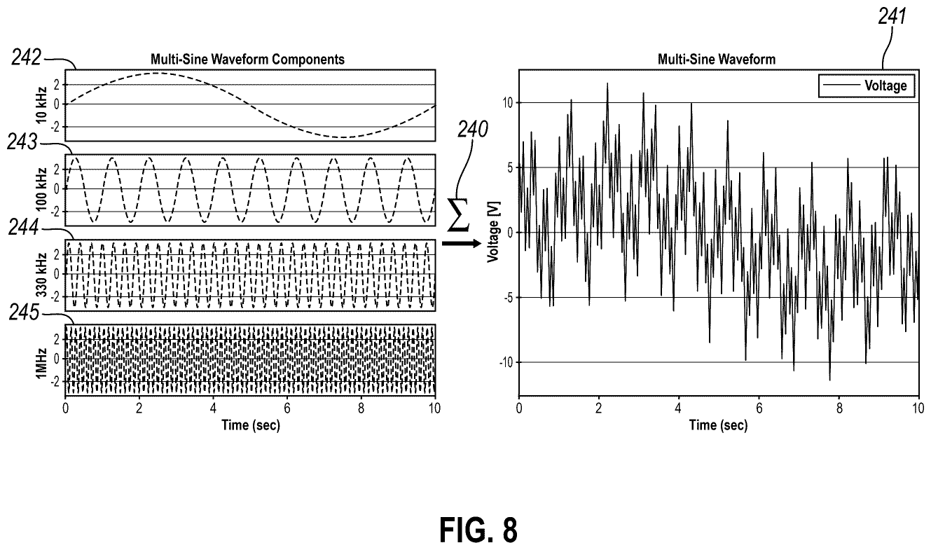

As discussed herein, the system may pass a non-therapeutic waveform through a portion of patient tissue to help identify the type of tissue as well as any foreign objects. Thus, in some versions, the system may pass waveforms of varying frequency (e.g., in series and/or parallel) to improve the accuracy of the determination. Accordingly, in some versions, and as shown in FIG. 8, multiple waveforms of various frequencies may be added or summed together (240) to create a muti-sine waveform (241). By way of non-limiting example, a 10 kHz sine wave (242) may be combined with a 100 kHz sine wave (243), a 330 kHz sine wave (244) and a 1 MHz sine wave (245) may be combined to create multi-sine wave (241).

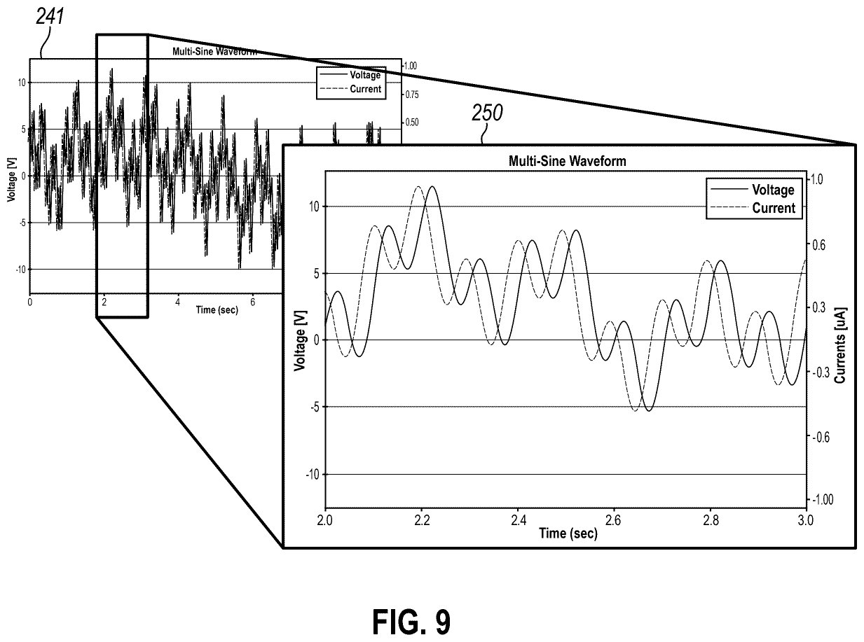

Referring now to FIG. 9, multi-sine waveform (241) may be sampled or windowed. In some versions, such as those that require the use of Fast Fourier Transforms (FFT), the windowing or sampling may be as small as a single period for the lower frequency waveform. As shown in a fourth graph (250), the voltage of multi-sine waveform (241) is leading the current and thus in the present version indicates a capacitive circuit (e.g., likely tissue). In an alternative version, the system may apply a series of burst waveforms having different frequencies.

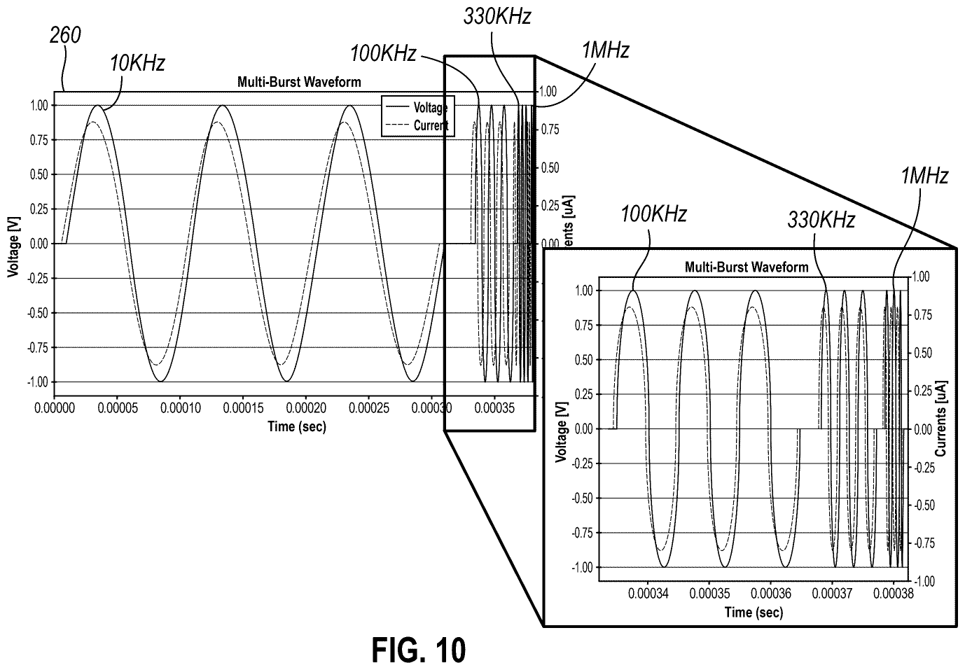

Referring now to FIG. 10, a burst waveform, including a brief delay between frequencies, is shown in fifth graph (260). In some versions, and as shown, the system may output a burst waveform that is a sine wave, while in other versions, the wave may be a square, triangle, ramp, pulse, pseudorandom binary sequence (PRBS), or arbitrary waveform. In some versions, the pause between waveforms can be evaluated in order to determine a “rebounding” time. The rebounding time may be used to help identify tissue types by evaluating how long certain tissues take to allow the waveform and any residual energy to dissipate from the tissue.

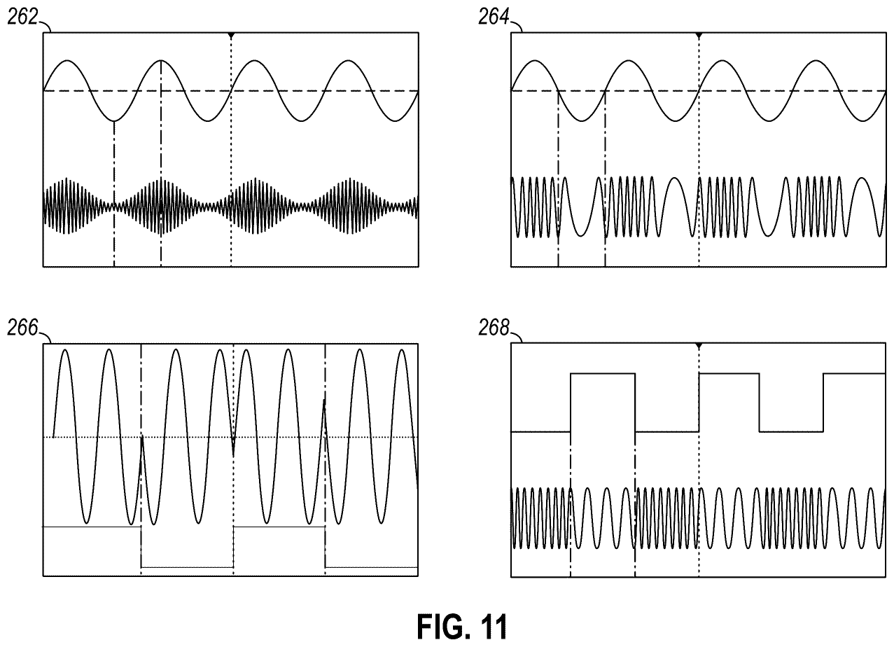

FIG. 11 shows various alternative burst versions. Specifically, in one version, amplitude modulation (AM) (262) may be used; while in another version, frequency modulation (FM) (264). Other versions may use phase modulation (PM) (266) and/or frequency-shift keying (FSK) modulation (268). Due to the fact that all of the modulation options shown in FIG. 11 involve a shift of some type, they may all be evaluated in a similar manner.

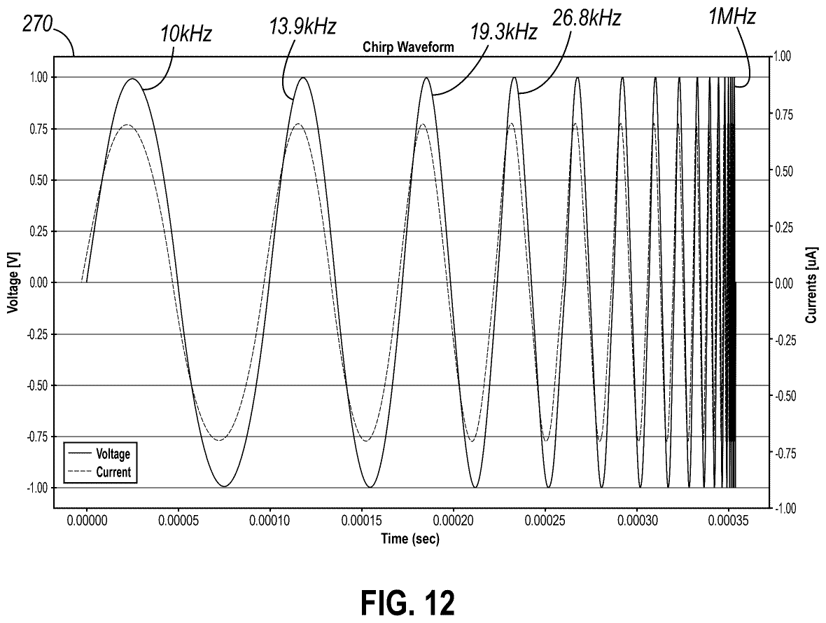

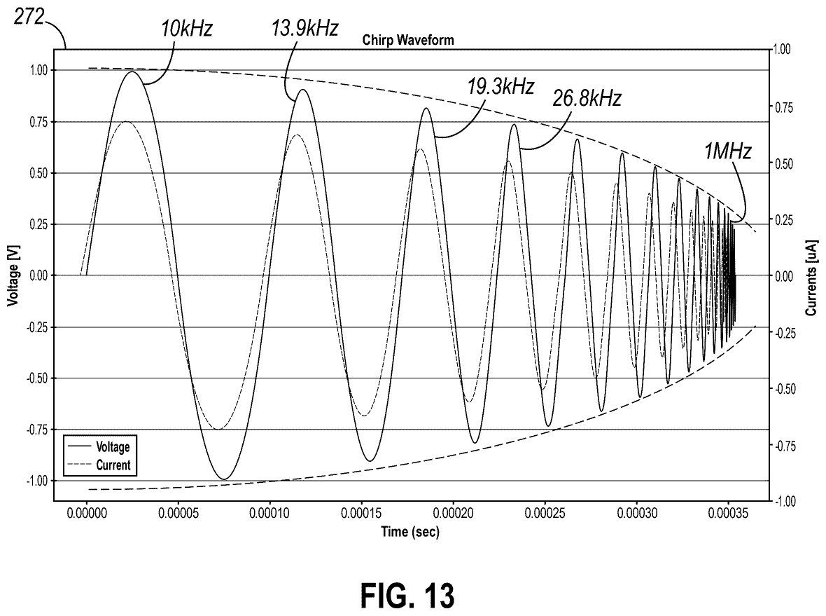

In a further version, a “chirp” function can be used, such as shown in FIG. 12. As would be understood by one skilled in the art, a chirp wave can be an “up-chirp” (i.e., the frequency increases) or a “down-chirp” (i.e., the frequency decreases). Thus, stated differently, a chirp function is essentially an advanced form of FM (264). The chirp function shown in a sixth graph (270) shows a chirp waveform with increasing frequency (e.g., 10 kHz, 13.2 kHz, 19.3 kHz, 26.8 kHz, and 1 Mhz). FIG. 13 shows a seventh graph (272) depicting a chirp function with the same frequencies as shown in FIG. 12, but with a decreasing amplitude. Additional features associated with electrical circuits and measurements of tissue are described in U.S. patent application Ser. No. 17/854,306, entitled “Electrosurgical Instrument for Applying Non-Therapeutic RF Signals,” filed Jun. 30, 2022, and published as U.S. Pat. Pub. No. 2024/0000499 on Jan. 4, 2024, the disclosure of which is incorporated by reference herein, in its entirety.

### III. Means of Identifying when Electrodes Require Cleaning

Surgical debris (such as eschar, dry blood, etc.) may accumulate on end effector (180), including electrodes (194, 196), of instrument (100) over the course of many activations during a procedure. Surface coatings on suitable portions of end effector (180), such as electrodes (194, 196), can inhibit accumulation/build-up of surgical debris thereon. Accumulation of surgical debris may occur at different rates depending on the conditions encountered. For example, the presence of significant amounts of blood during the procedure may increase the rate of buildup on the electrodes (194, 196).

Accumulation of surgical debris on end effector (180) may decrease the rate of heat generation during activation, which may have a negative impact on electrodes (194, 196) suitably sealing grasped tissue via therapeutic RF energy. Additionally, accumulation of surgical debris on end effector (180) may increase measured impedance at electrodes (194, 196). Cleaning end effector (180) can remove surgical debris bound to the electrodes and restore performance. Therefore, it may be desirable to determine when end effector (180) requires cleaning, notify a user that end effector (180) requires cleaning, and/or modify the therapeutic energy delivery algorithm in order to accommodate for an end effector (180) with an undesirable amount of tissue debris accumulated thereon.

A. Electrosurgical Instrument with Subtherapeutic Impedance Sensing in Open-Jaw to Detect Dirty Jaws

As mentioned above, end effector (180) is configured to apply non-therapeutic bipolar radiofrequency (RF) energy to securely clamped tissue in order to identify and/or verify that the correct tissue is presented in end effector (180) such that a therapeutic RF energy can be applied to seal or weld tissue. In the current example, electrosurgical instrument (100) is electrically coupled to waveform generator (200), which is capable of delivering both therapeutic and non-therapeutic energy to end effector (180). Non-therapeutic energy may also be referred to as subtherapeutic energy.

Further, one or more electrodes (194, 196) of end effector (180), which are configured to engage tissue when end effector (180) clamps tissue in accordance with the description herein, are operatively connected to sensor devices to determine the impedance phase angle and magnitude of the electrical circuit which non-therapeutic energy is transmitted through (e.g., such an electrical circuit may be partially formed by electrodes (194, 196) and the tissue captured therebetween). A processor (e.g., control unit (102)) may receive this impedance information, utilize the impedance information, and/or relay such impedance information to suitable electronic devices for use during or after a surgical procedure. As also mentioned above, electrodes (194, 196) may be activated with therapeutic RF energy in order to seal or weld tissue captured between jaws (182, 184), while knife member (176) may be utilized in order to sever tissue captured between jaws (182, 184).

In some instances, it may be desirable to transmit non-therapeutic energy to one or more electrodes (194, 196) and measure the resulting impedance phase angle and magnitude of the corresponding electrical circuit during moments other than when tissue is securely clamped by end effector (180). Applying non-therapeutic energy to one or electrodes (194, 196) and measuring the resulting impedance phase angle and magnitude of the corresponding electrical circuit during other moments of a surgical procedure may provide other insights. Such insights may be utilized to inform a surgeon, and/or may be logged into data storage for later use. Applying non-therapeutic energy to one or more electrodes (194, 196) and measuring the resulting impedance phase angles and magnitudes of the corresponding electrical circuit may be referred to as impedance sensing. In some instances, non-therapeutic energy may be applied to one or more electrodes (194, 196) at multiple frequencies such that impedance phase angles and magnitudes are measured for each frequency.

During illustrative use of end effector (180), buildup of surgical debris may undesirably accumulate and stick to the surfaces of jaws (182, 184) and/or electrodes (194, 196). This undesirable accumulation of surgical debris on end effector (180) can result in jaws (182, 184) undesirably sticking together (e.g., not suitably opening), premature termination of energy delivery algorithms, and/or shunting energy away from the tissue to be sealed. These results may pose a risk to seal quality. Accumulation of surgical debris may also limit the capability of sensing technologies implemented on end effector (180), such as bioimpedance spectroscopy, or optical sensing modalities, as the sensor measurements will be influenced by material built up on the electrodes in addition to the target tissue to seal.

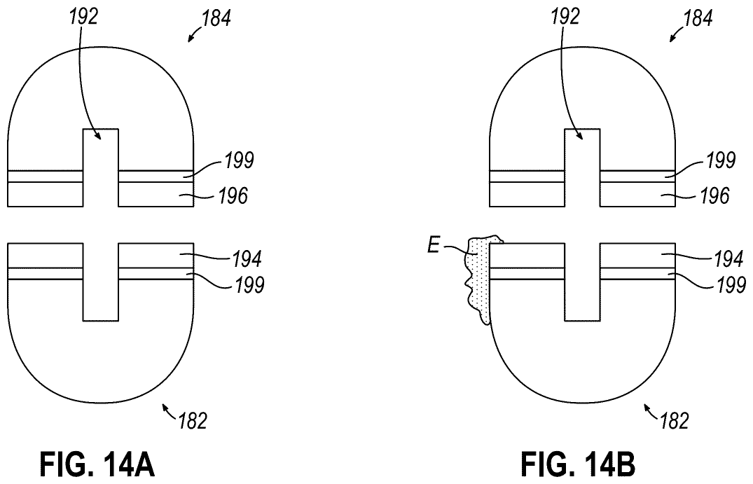

FIG. 14A shows a clean end effector (180) with jaws (182, 184) in the open position. As can be seen in FIG. 14A, since end effector (180) is clean, there is little to no surgical debris accumulated on the surfaces of jaws (182, 184) and/or electrodes (194, 196). FIG. 14B shows end effector (180) of electrosurgical instrument (100) with jaws (182, 184) in the open position after a plurality of energy activation cycles to seal tissue captured between electrodes (194, 196) and/or knife firings to sever tissue captures between electrodes (194, 196).

While jaws (182, 184) are in the open position and clean, as illustrated in FIG. 14A, the measured impedance magnitude from impedance sensing may be very high. As mentioned above, impedance sensing may include activating one or more electrodes (194, 196) with non-therapeutic energy and measuring the resulting impedance phase angle and magnitude of the corresponding electrical circuit. As best shown in FIGS. 14A-14B, electrodes (194, 196) are intended to be electrically insulated from their respective jaw (182, 184) (and other suitable components of end effector (180)) via a suitable insulating material (199). Electrodes (194, 196) are electrically insulated from other suitable components of end effector (180) such that electrodes (194, 196) may suitably transmit therapeutic and/or non-therapeutic energy to tissue grasped between jaws (182, 184). The high impedance magnitude measurement of a clean end effector (180) (as shown in FIG. 14A) may be because electrodes (194, 196), while in the open position, are effectively forming an open circuit.

However, as illustrated in FIG. 14B, after end effector (180) suitably seals and severs tissue, surgical debris (e.g., eschar (E)) may accumulate on the surfaces of end effector (180). Accumulation of surgical debris may create a current path between electrode (194, 196) and its corresponding jaw (182, 184) (or other components of end effector (180)), effectively bypassing the insulating material (199) interposed between the two. In the present illustrative example, eschar (E) has accumulated on lower jaw (182) and electrode (194), thereby forming a current path between lower jaw (182) and electrode (194). Therefore, if impedance sensing occurs while jaws (182, 184) are in the open position as shown in FIG. 14B, the measured “open jaw” impedance magnitude may be lower as compared to when end effector (180) is substantially clean as shown in FIG. 14A. In other words, a reduction in measured impedance magnitude from impedance sensing while jaws (182, 184) are in the open position may occur at least in part due to unintended current paths formed by electrodes (194, 196) and other components of instrument (100) via accumulated surgical debris.

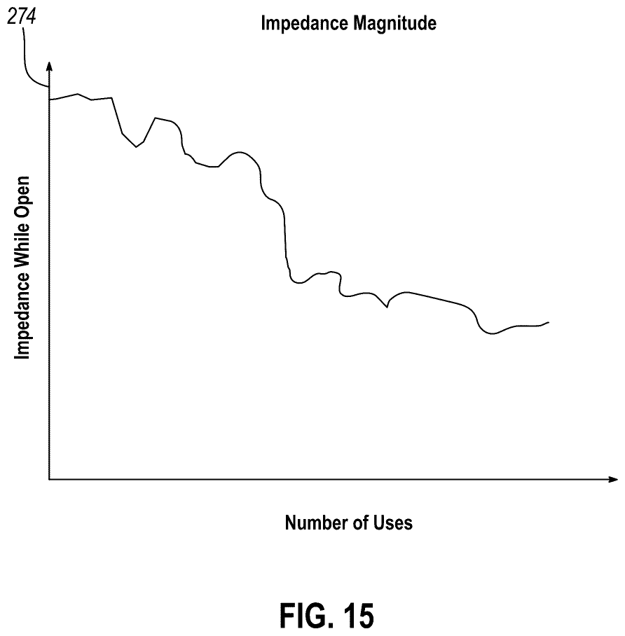

Further, as more surgical debris accumulates, the measured impedance magnitude from impedance sensing while jaws (182, 184) are in the open position may continue to decline. FIG. 15 is a graph (274) displaying an illustrative measured impedance magnitude of jaws (182, 184), while in the open position, from impedance sensing compared to the number of activations and/or knife firings of end effector (180) to seal and/or sever tissue in accordance with the description herein. As can be seen, the measured open jaw impedance magnitude generally decreases as the number of activations and/or knife firings increases.

Therefore, it may be desirable to provide a means to determine if end effector (180) has an undesirable accumulation of surgical debris during illustrative use of instrument (100). Additionally, it may be desirable to alert users of such an undesirable accumulation of surgical debris, such that users may clean end effector (180) during illustrative use in accordance with the description herein. Additionally, or alternatively, it may be desirable to alter the energy delivery algorithm to compensate for accumulated surgical debris on end effector (180) and maintain performance of end effector (180). Due to the above described trend in impedance sensing magnitude v. the number of activations and/or knife firings, subtherapeutic impedance sensing of jaws (182, 184) in the open position, between activations of end effector (180), may be a potential means to detect an accumulation of surgical debris in order to response appropriately.

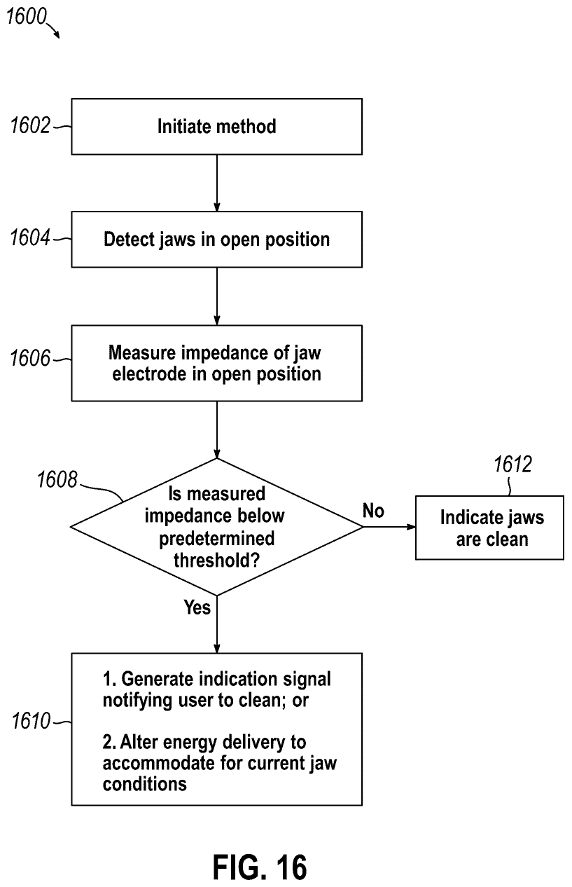

FIG. 16 shows a flowchart depicting an illustrative inspection method (1600) performed by surgical system (98) to determine if end effector (180) has accumulated an undesirable amount of surgical debris during illustrative use, and reacting accordingly. First, illustrative method (1600) may start by initiating (1602) the inspection method (1600). In some instances, surgical system (98) may initiate method (1602) automatically in response to another suitable event as would be apparent to one skilled in the art in view of the teachings herein. In some instances, inspection method (1600) is initiated by a user providing instructions to surgical system (98) (e.g., via waveform generator (200), via a suitable interface on instrument (100), or via another suitable user interface) to start method (1600).

As one example, during a surgical procedure, a surgeon may inquire whether or not end effector (180) has accumulated an undesirable amount of surgical debris such that surgeon removes instrument (100) from a patient to inspect jaws (182, 184). A user may instruct surgical system (98) to initiate method (1600) once instrument (100) is removed from the patient and/or instrument (100) is being prepared to be removed from the patient. It may be desirable to remove end effector (180) from patient prior to moving forward with the inspection method (1600), as jaws (182, 184) may be prone to engage with the surgical environment (i.e., fluids, tissue, etc.) while remaining within the patient, which may inadvertently affect the quality of data accumulated during inspection method (1600).

One inspection method (1600) has been initiated (1602), system (98) may then wait to detect (1604) if jaws (182, 184) are in the open position. Jaws (182, 184) may already be in the open position after being suitably removed from a patient. Alternatively, a user may open jaws (182, 184) in accordance with the teachings herein. In some instances, system (98) may detect (1604) that jaws are in the open position by waiting for user input confirming that jaws (182, 184) are in the open position. In other instances, instrument (100) may include suitable components that may be utilized to assist system (98) in detecting that jaws (182, 184) are in the open position. In some embodiments, this detection could come from continuous jaw aperture measurement using a suitable jaw gap sensor as would be apparent to one skilled in the art in view of the teachings herein. For example, a suitable jaw gap sensor may include a hall-effect sensor. In other embodiments, a switch in the handle assembly (120) may detect when jaws (182, 184) are clamped or open. In further embodiments still, impedance sensing may detect the signature of an open jaw.

End effector (180) may include any suitable components to perform impedance sensing as would be apparent to one skilled in the art in view of the teaching herein. For example, the electrical circuit measured between electrodes (194, 196) may be utilized in impedance sensing. In other examples, dedicated sensing electrodes, such as using measurements between two sensing locations on electrode (194) to detect current path associated with built-up surgical debris on the surface of electrode (194).

Once system (98) detects (1604) jaws (182, 184) are in the open position during detection method (1600), system (98) measures (1606) the impedance magnitude of suitable jaw electrodes (e.g., one or more electrodes (194, 196), dedicated impedance sensing electrodes, etc.). Therefore, it should be understood that system (98) instructs waveform generator (200) to apply non-therapeutic RF energy to end effector (180) and measure the resulting impedance magnitudes and phase angles. It should be understood that this impedance measurement (1606) occurs while jaws (182, 184) are in the open position. System (98) may utilize any suitable components (e.g., control unit (102), waveform generator (200), and/or other suitable electrical components) to measure the resulting impedance magnitude(s) and phase angle(s) as would be apparent to one skilled in the art in view of the teachings herein.

Next, system (98) may compare the measured (1606) impedance magnitude with a predetermined threshold. Such a predetermined threshold may be a suitable impedance magnitude(s) value that is associated with jaws (182, 184) being suitably free from surgical debris such that end effector (180) may satisfactorily function in accordance with the teachings herein. Any suitable threshold value and/or range may be utilized as would be apparent to one skilled in the art in view of the teachings herein.

Therefore, if the measured (1606) impedance magnitude is above the predetermined threshold, system (98) may indicate (1612) to a user that jaws (182, 184) are suitably clean and do not necessarily require further cleaning. Such an indication (1612) may include visual and/or audible feedback and may include any suitable message as would be apparent to one skilled in the art in view of the teachings herein.

However, if the measured (1606) impedance magnitude is below the predetermined threshold, system (98) may generate and indication signal (1610-1), indicating to a user that jaws (182, 184) need to be cleared; and/or system (98) may alter the energy deliver algorithm (1610-2) in order to accommodate for the current jaw conditions. System (98) may use any suitable type of alert signal as would be apparent to one skilled in the art in view of the teachings herein. For example, a visual and/or audio signal may be generated. Subsequently, a user may clean jaws (182, 184) in order to remove accumulated surgical debris. After cleaning of jaws (182, 184), system (98) and or a user may have system (98) rerun method (1600) in order to ensure jaws (182, 184) are suitably clean (e.g., the subsequent impedance magnitude measurement is above the predetermined threshold). Alternatively, a user may simply indicate to system (98) that jaws have been cleaned and are ready for illustrative use in accordance with the teachings herein. Alternatively, if a user elects to not clean jaws (182, 184) the therapeutic energy delivery algorithm may be altered in order to accommodate for the accumulated surgical debris.

### B. Jaw-Gap Measurement and Tissue Sticking

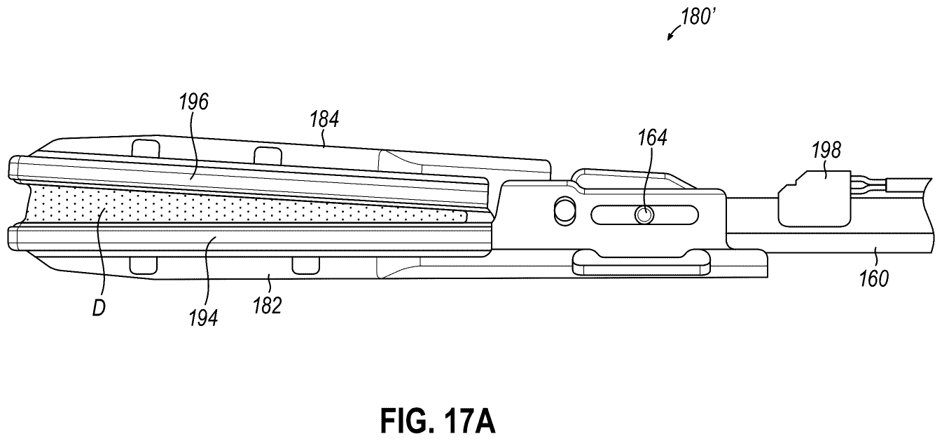

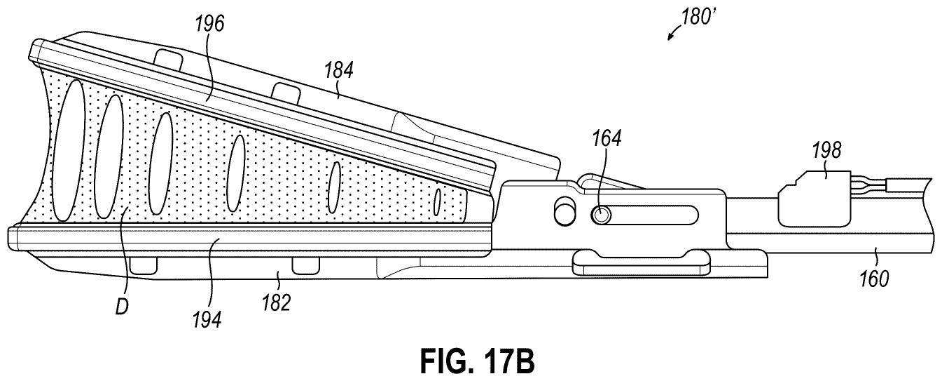

As previously mentioned above, an undesirable accumulation of surgical debris on jaws (182, 184) of end effector (180) may result in jaws (182, 184) undesirably sticking together (e.g., not suitably opening). When surgical debris accumulates on the surfaces of electrodes (194, 196), such surgical debris may be interposed between electrodes (196, 194) such that when jaws (182, 184) attempt to open (as illustrated between FIGS. 17A-17B), the interposed surgical debris (D) remains engaged with each electrode (194, 196) (or other components of end effector (180)), thereby inhibiting jaws (182, 184) from pivoting away from each other. The presence of such surgical debris (D) may cause jaws (182, 184) to open at a slower rate as compared to instances where surgical debris (D) is not present between electrodes (194, 196).

Therefore, it may be desirable to monitor the rate at which jaws (182, 184) transition from the closed position toward the open position. Further, if it is determined that the opening rate of jaws (182, 184) is below a predetermined threshold, it may be desirable to indicate to a user that jaws (182, 184) need to be cleaned based, at least in part, on the monitored opening rate. Additionally, or alternatively, if it is determined that the opening rate of jaws (182, 184) is below a predetermined threshold, it may be desirable to warn a user to take precautions when opening jaws (182, 184) to release grasped tissue. Additionally, or alternatively, if it is determined that the opening rate of jaws (182, 184) is below a predetermined threshold, it may be desirable to alter the energy delivery algorithm to accommodate for such accumulated surgical debris in order to ensure tissue captured between jaws (182, 184) suitably seals if jaws (182, 184) are not cleaned prior to further illustrative use.

FIGS. 17A-17B show an alternative end effector (180′) that may be readily incorporated into surgical instrument (100) in replacement of end effector (180) described above. Therefore, end effector (180′) may be substantially similar to end effector (180) described above, with differences elaborated below. In particular, end effector (180′) includes a jaw gap sensor (197). Jaw gap sensor (197) is configured to measure a jaw aperture (e.g., jap gap) between jaws (182, 184). Further jaw gap sensor (197) may be utilized to determine the opening rate at which jaws (182, 184) move relative to each other when transitioning from a closed position (see FIG. 17A) into the opened position (see FIG. 17B). Jaw gap sensor (197) is in suitable communication with control unit (102). Jaw gap sensor (197) may be in suitable communication with control unit (102) via any suitable structures as would be apparent to one skilled in the art in view of the teachings herein.

Jaw gap sensor (197) in the current example is suitably attached to jaw closure connector (160). As mentioned above, jaw closure connector (160) is configured to translate in order to open and close jaws (182, 184) in accordance with the teaching herein. Jaw gap sensor (197) may measure the displacement of jaw closure connector (160) and/or rate of change (e.g., velocity and/or acceleration) of jaw closure connector (160). Further, jaw motion sensor (197) may communicate these measurements to control unit (102). Since proximal movement of jaw closure connector (160) corresponds to the opening rate of jaws (182, 184), control unit (102) may utilize the measurements received from jaw gap sensor (197) to determine the opening rate of jaws (182, 184) based at least in part on the proximal displacement, movement, and/or acceleration of jaw closure connector (160).

Jaw gap sensor (197) may include any suitable structures as would be apparent to one skilled in the art in view of the teachings herein. For example, jaw gap sensor (197) may include a hall effect sensor, a linear displacement sensor, etc. While jaw gap sensor (197) is shown attached to jaw closure connector (160) in the current example, it should be understood that jaw closure connector (160) maybe attached to any suitable structures as would be apparent to one skilled in the art in view of the teachings herein. For example, jaw gap sensor (197) may include an inclinometer attached to jaws (182, 184) and configured to directly measure the angle formed between jaws (182, 184).

FIGS. 17A-17B show an illustrative opening of jaws (182, 184) with tissue debris (D) interposed between electrodes (194, 196). As jaws (182, 184) open, the rate that jaws (182, 184) open will be influenced by the severity of debris and/or eschar (D) built up between jaws (182, 184). As mentioned above, sensor (197) measures the opening rate of jaws (182, 184) in order to evaluate the severity of debris/eschar (D) buildup in jaws (182, 184). As also mentioned above, the more sever the debris/eschar buildup (D), the slower jaws (182, 184) will open compared to if no debris accumulated.

Sensor (197) further communicates the measured rate of opening of jaws (182, 183) (or other suitable monitored parameters that enable a suitable calculation of rate of opening) with suitable components of surgical system (98), such as control unit (102), waveform generator (200), or other suitable electronic components as would be apparent to one skilled in the art in view of the teachings herein. Surgical system (98) may compare the measured rate of opening to a predetermined threshold rate of opening. Based off such comparison, surgical system (98) may utilize this information to determine if jaws (182, 184) should be cleaned, if a user should be warned, and/or if a therapeutic energy delivery algorithm should be modified.

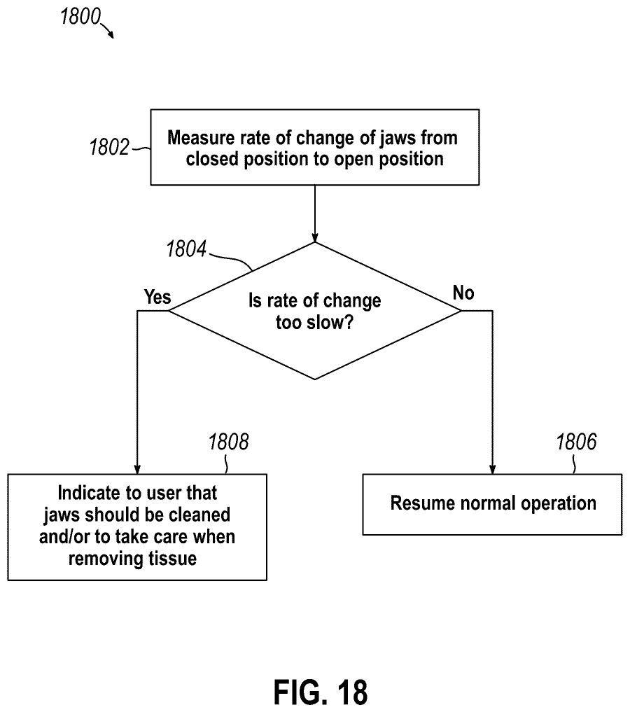

FIG. 18 shows a flowchart depicting an illustrative method (1800) of measuring the opening rate of jaws (182, 184) from the closed position to the open position in order to determine whether or not to clean jaws (182, 184), warn a user, and/or alter the therapeutic energy delivery algorithm. First, suitable components of system (98) may measure (1802) the opening rate of change of jaws (182, 184) transitioning from the closed position (see FIG. 17A) to the opened position (see FIG. 17B). Measurements (1802) may be communicated from sensor (197) to suitable electrical components of system (98), such as control unit (102), waveform generator (200), or other suitable components. It should be understood that method (1800) may be initiated and performed used at any suitable time during a surgical procedure as would be apparent to one skilled in the art in view of the teachings herein. For instance, method (1800) may be initiated while end effector (180) is positioned within a patient, when end effector (180) is temporarily removed from the patient, etc.

Next, the electrical components receiving measurement (1802) from sensor (197) may compare (1804) the measured opening rate of jaws (182, 184) with a predetermined threshold to determine if the rate of change is too slow. If the rate of change is not too slow, this may indicate an electrode (194, 196) are suitably free from surgical debris in order to desirably function in accordance with the description herein. As such, system (98) may resume (1806) normal operation. In some instances, system (98) may inform a user that jaws (182, 184) are opening at a sufficient rate. In other instances, system (98) may not inform a user that jaw s9812, 184) are opening at a sufficient rate.

However, if the rate of change is determined to be too slow, system (98) may indicate (1808) to a user that jaws should be cleaned, that a user should take care while opening jaws (182, 184) to release grasped tissue, and/or system (98) may alter the therapeutic energy delivery algorithm to accommodate for the additional debris (D). Therefore, system (98) may utilize sensor (197) in order to alert a user of such accumulated debris and/or modify illustrative use of instrument (100) to account for such accumulated debris.

### C. Jaw Angle Sensing Application for Tissue Buildup

As mentioned above, jaw closure trigger (126) may be pivoted toward pistol grip (124) in order to pivot jaws (182, 184) from an open position into a clamped position. As also mentioned above, end effector (180′) includes a suitable jaw gap sensor (197) that is configured to measure a jaw gap between jaws (182, 184) during illustrative use, and communicate the measured jaw gap to suitable electrical components of system (98) (e.g., control unit (102)).

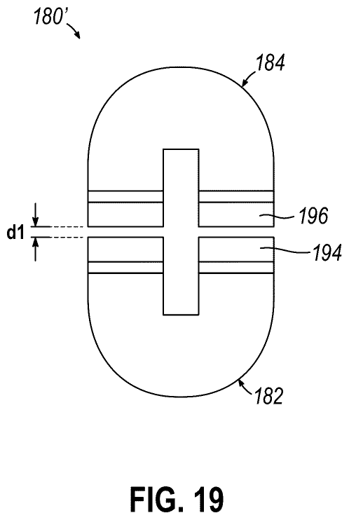

When jaws (182, 184) are clean and free from surgical debris, electrodes (194, 196) may define a known gap distance (d1) (see FIG. 19) while jaws (182, 184) are in the clamped position with nothing interposed between jaws (182. 184) (e.g., there is no tissue between jaws (182, 184)). Such a known gap distance (d1) may be referred to as a baseline gap distance (d1). The baseline gap distance (d1) may be stored on suitable electrical components of system (98) (e.g., control unit (102), waveform generator (200), etc.) and used as a baseline reference. Such a known gap distance (d1) may be determined prior to initial use of end effector (180′) and/or during the manufacturing process of end effector (180′). In some cases, gap distance (d1) may be determined at the start and/or finish of each procedure. Of course, the baseline gap distance (d1) may be determined at any other suitable time as would be apparent to one skilled in the art in view of the teachings herein.

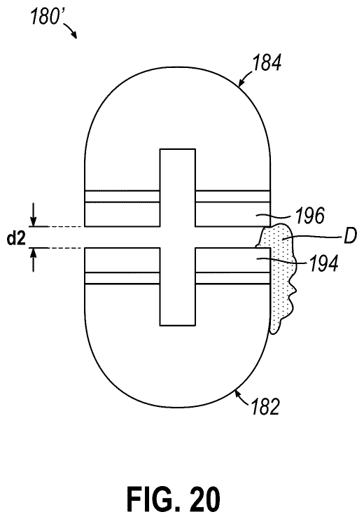

However, in instances where surgical debris accumulates on the surface of electrodes (194, 196) during illustrative use of end effector (180′) in accordance with the description herein, excessive debris buildup (D) (see FIG. 20) may inhibit jaws (182, 184) from defining the predetermined gap distance (d1) while in the clamped position with nothing interposed therebetween. Instead, jaws (182, 184) may define a second gap distance (d1) (see FIG. 20) when pivoted into the clamped position with nothing therebetween, which is greater than the predetermined gap distance (d1) (see FIG. 19).

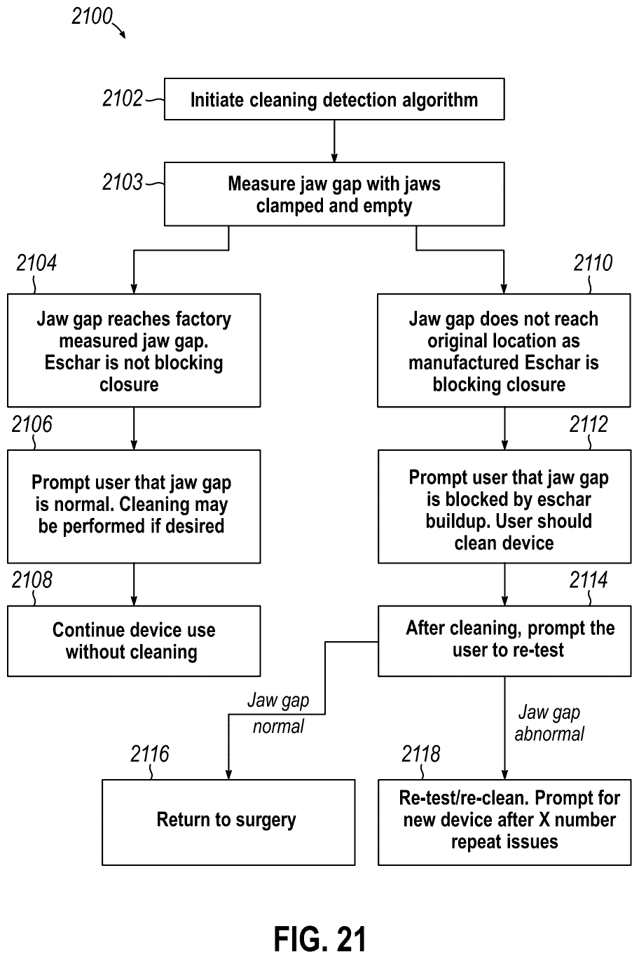

Therefore, it may be desirable to use jaw gap measurements to determine when excessive surgical debris/eschar has built up in between the jaws (182, 184) of end effector (180′). FIG. 21 shows a flowchart of an illustrative cleaning detection algorithm (2100) measuring a jaw gap between jaws (182, 184) in the clamped position with nothing interposed therebetween, and comparing the measured jaw gap with a baseline jaw gap in order to determine if end effector (180′) should be cleaned. First, a cleaning detection algorithm is initiated (2102). This may commence when the user selects a prompt to initiate (2102) cleaning detection algorithm. Of course, cleaning detection algorithm may be initiated (2102) via any other suitable manner as would be apparent to one skilled in the art in view of the teachings herein. For example, a prompt to initiate (2102) the cleaning detection algorithm may be generated by system (98) in response to system (98) detecting electrical impedance data that is indicative of eschar being present on jaws (182, 184).

Next, a user may be prompted to pivot jaws (182, 184) into the clamped position while jaws (182, 184) are empty (i.e., there are easily removable foreign objects present between electrodes (194, 196) and/or other suitable portions of jaws (182, 184)). A user may confirm that jaws (182, 184) have been pivoted into the clamped position. Additionally, or alternatively, instrument (100) incorporated with end effector (180′) may include a clamp switch that communicates with control unit (102) when jaws (182, 184) are clamped. It should be understood that at this moment, end effector (180′) may be located within patient during a surgical procedure or may be removed from patient. After the user pivots jaws (182, 184) into the emptied, clamped position, system (98) may measure (2103) the jaw gap while jaws (182, 184) are both clamped and empty. For example, jaw gap senor (197) may be utilized to measure the jaw gap while jaws (182, 184) are in the clamped position and empty. Sensor (197) may communicate this measurement to other suitable electrical components of system (98), such as control unit (102), waveform generator (200), or other suitable components as would be apparent to one skilled in the art in view of the teachings herein.

If system (98) determines that the measured (2103) jaw gap has reached (2104) the baseline jaw gap (d1) (or at least within a predefined range of the baseline jaw gap (d1), system (98) may prompt (2106) a user that the jaw gap is normal and/or the cleaning is not required, but may be performed in desired. Therefore, a user may continue (2108) to use device without cleaning end effector (180′). In the current example, the baseline jaw gap (d1) is the factory measured jaw gap. However, as mentioned above, the baseline jaw gap (d1) may be defined at any other suitable time as would be apparent to one skilled in the art in view of the teachings herein.

If instead, system (98) determines that the measured (2103) jaw gap does not reach (2110) the baseline jaw gap (d1) (or at least within a predefined range of the baseline jaw gap (d1)), system (98) may prompt (2112) a user that the jaw gap is not normal (e.g., blocked by eschar buildup) and/or that end effector (180′) should be cleaned. Once a user indicated to system (98) that end effector (180′) has been cleaned, system (98) may prompt (2114) a user to re-test via cleaning detection algorithm. If the re-test shows jaw gap is back within the desired range, system (98) may return to surgery (2116). If instead system (98) measures an abnormal jaw gap, system (98) will prompt (2118) the user to re-test jaw gap or re-clean jaws (1982, 1984).

In some embodiments, if re-testing keeps indicating that jaw gap is not within a desired range, system (98) will prompt (2118) the user to retrieve a new device.

### D. Dirty Jaw Detection by Tracking Trends in Impedance During Therapeutic Activation Cycle

As mentioned above, impedance sensing using non-therapeutic RF energy may be utilized while jaws (182, 184) are in the open position to determine if jaws (182, 184) need to be cleaned during illustrate use of instrument (100). However, impedance measurements other than using non-therapeutic RF energy when jaws are in the open position may be utilized in order to determine if jaws (182, 184) need to be cleaned. For example, impedance magnitudes associated with electrodes (194, 196) being activated with therapeutic RF energy to seal tissue may be measured and stored on suitable components of system (98) during illustrative use of instrument (100) for a surgical procedure. As will be described in greater detail below, such impedance data may be utilized to determine if end effector (180, 180′) needs to be cleaned.

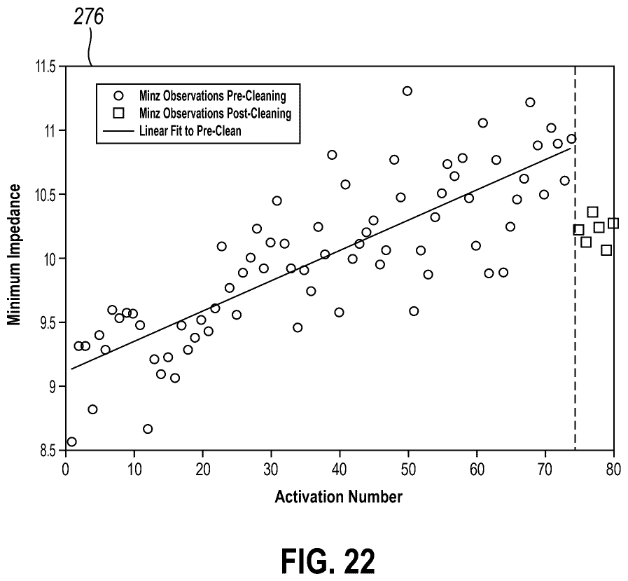

FIG. 22 shows a scatterplot graph (276) tracking the minimum impedance magnitude measured and recorded for each activation cycle of end effector (180) over the course of the use of instrument (100). At the start of a surgical procedure, the minimum impedance magnitude recorded during RF energy activation cycle is relatively low. However, the minimum impedance magnitude will gradually increase as more activations are performed and surgical debris builds up on the electrodes (194, 196). The proteins in blood will bind readily to electrodes (194, 196), so if blood is encountered during surgery, tissue buildup may occur at a much faster rate compared to a procedure that encounters less blood. A plot of minimum impedance magnitude from each activation will appear noisy due to differences in tissue from activation to activation. Therefore, a method of smoothing the data by either fitting a polynomial or filtering the data may be utilized.

As will be described in greater detail below, if the filtered minimum impedance magnitude value for an activation cycle surpasses a predetermined threshold, this may be indicative that end effector (180) needs to be cleaned. Such a predetermined threshold may be determined though any suitable means as would be apparent to one skilled in the art in view of the teachings herein. For example, the closed loop impedance of the device may be measured on the manufacturing line. This measured closed loop impedance may be used to create a predetermined threshold. As another example, the predetermined threshold may be calculated based on an initial therapeutic activation cycle(s) of a surgical procedure.

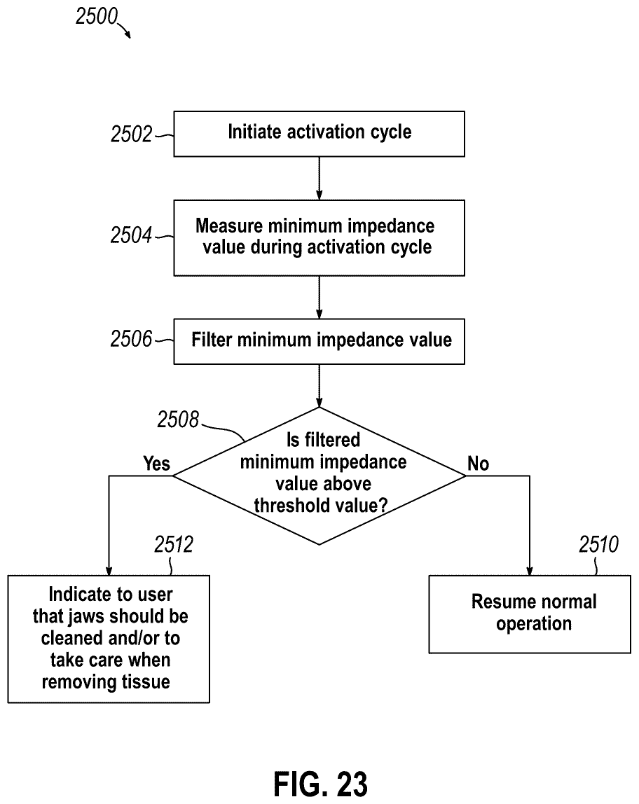

FIG. 23 shows a flowchart depicting a method (2500) of how minimum impedance magnitude can be used by system (98) to determine whether to clean jaws (182, 184). First, an activation cycle is initiated (2502). Then, system (98) measures (2504) the minimum impedance magnitude during a therapeutic RF energy activation cycle of end effector (180) in order to seal tissue located adjacent to electrodes (194, 196). The measured minimum impedance magnitude value is then filtered (2506) and stored using suitable electric devices of system (98) (e.g., control unit (102), waveform generator (200), etc.). Next, the system (98) asks (2508) if the filtered minimum impedance magnitude value is above the predetermined threshold. If the filtered minimum impedance magnitude value is above a certain threshold, system (98) indicates (2512) to the user that jaws (182, 184) should be cleaned. In some embodiments, a warning on a screen or an audio alert can be used to inform the staff that jaws (182, 184) need to be cleaned. In some embodiments, waveform generator (200) also alerts the user to take care when removing tissue. If the filtered minimum impedance magnitude value is not above a certain threshold, system instructs the user to resume (2510) normal operation.

In the current example, the minimum impedance magnitude value measured during each therapeutic energy activation cycle is used. However, this is merely illustrative. Any other suitable metric obtained during a therapeutic energy activation cycle may be used as would be apparent to one skilled in the art in view of the teachings herein. For example, the starting impedance magnitude, the average impedance magnitude, or the ending impedance magnitude may be used.

Additionally, in the current example, each individual minimum impedance magnitude value per therapeutic RF energy activation cycle is compared against the predetermined threshold in order to determine if jaws (182, 184) need to cleaned. However, as will be described in greater detail below, impedance data collected over a span of activation cycles may be averaged and compared to the predetermined threshold in order to determine if jaws (182, 184) require cleaning.

It should be understood that in some instances, the measured impedance magnitude value during a therapeutic RF energy activation cycle may actually decrease over the lifespan of a device. This may occur due to a reduction in jaw gap due to a wear down of jaws (182, 184). In particular, if, instead of a jaw (182, 184) having a corresponding electrode (194, 196), a jaw (182, 184) may include a tissue pad that wears over time. Additionally, or alternatively, some tissue or liquid may be located on the proximal end of jaws (182, 184) and may draw current, which also may reduce a measured impedance magnitude value. Deviation of measured impedance magnitude value over the lifespan of instrument (100) too far in either direction (above or below) may be indicative of poor sealing performance. Therefore, in some instances, as will also be described in greater detail below, action may be taken if measured impedance data during therapeutic energy activation cycle(s) drops below a lower threshold. Such a lower predetermined threshold may be calculated using any suitable means as would be apparent to one skilled in the art in view of the teachings herein.

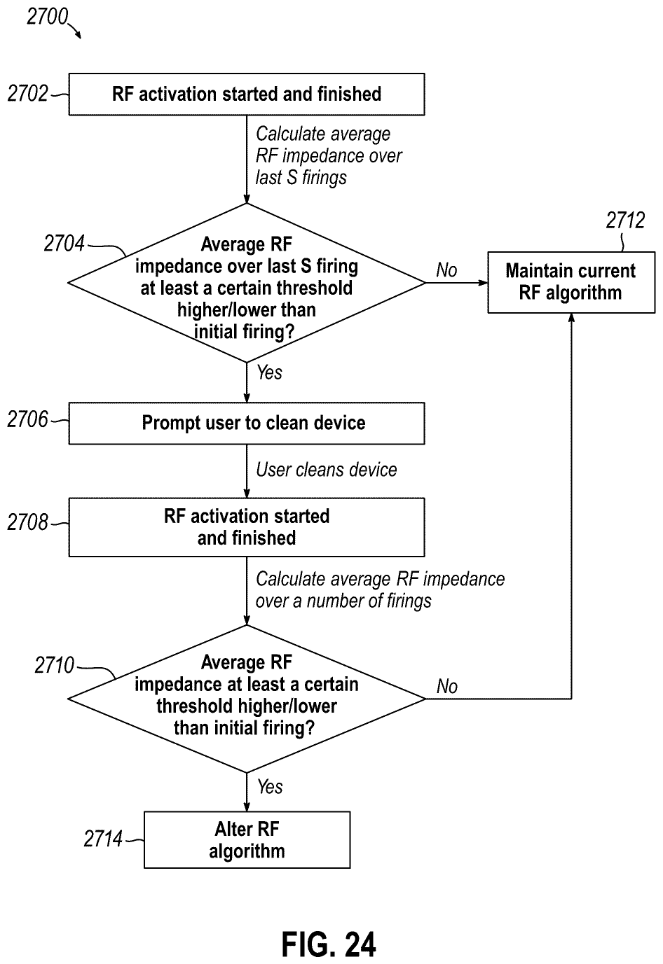

FIG. 24 shows a flowchart of another illustrative method (2700) using impedance data acquired during a therapeutic energy activation cycle can be used by system (98) to determine whether to clean jaws (182, 184). First, instrument (100) may complete (2702) an RF therapeutic energy activation cycle. During the RF therapeutic energy activation cycle, an impedance parameter (e.g., average impedance magnitude, starting impedance magnitude, ending impedance magnitude, minimum impedance magnitude, etc.) may be measured and stored by suitable electrical components of system (98) (e.g., control unit (102), waveform generator (200), etc.). In the current example, average impedance magnitude is measured. Therefore, the impedance magnitude is measured over the entirety of the RF therapeutic energy activation cycle, and then averaged over the timespan of the RF therapeutic energy activation cycle. For example, if an RF therapeutic energy activation cycle was two seconds long, and the measured impedance magnitude was 1 ohm for the first second of activation and 3 ohms for the remaining time of the RF therapeutic energy activation cycle, the average impedance would be 2 ohms. After the RF therapeutic energy activation cycle, system (98) then calculates a mean average impedance magnitude value over the last five RF therapeutic energy activation cycles to calculate a mean average impedance magnitude value.

As mentioned above, in some instances, measured impedance parameters may increase or decrease relative to a baseline, starting value. If the measured impedance parameters deviate too far from the baseline value, this may be indicative of less than desirable sealing performance. Therefore, after system (98) calculates the mean average impedance magnitude, system (98) then compares (2704) the mean average impedance magnitude value with both the upper predetermined threshold and the lower predetermined threshold. If system (98) determines that the mean average impedance is within the range defined by the upper and lower predetermined thresholds (i.e., a value simultaneously above the lower predetermined threshold and below the upper predetermined threshold), system may maintain (2712) the current algorithm for the RF therapeutic energy activation cycle.

However, if system (98) determines that the mean average impedance magnitude is outside the range defined by the upper and lower predetermined thresholds, system (98) may prompt (2704) a user to clean end effector (180). Such a prompt (2704) may be substantially similar to other prompts to clean end effector (180) described herein. After the user cleans end effector (180), instrument (100) may be sequentially activated to seal and/or sever tissue in accordance with the description herein.

Therefore, instrument (100) may complete (2708) an RF therapeutic energy activation cycle. Similarly, during the RF therapeutic energy activation cycle, an impedance parameter (e.g., average impedance, starting impedance, ending impedance, minimum impedance, etc.) may be measured and stored by suitable electrical components of system (98) (e.g., control unit (102), waveform generator (200), etc.). After the RF therapeutic energy activation cycle, system (98) then recalculates a mean average impedance magnitude value over the last five RF therapeutic energy activation cycles to calculate a mean average impedance magnitude value. System (98) again compares the newly calculated mean average impedance magnitude value to the upper and lower thresholds. If system (98) determines that the mean average impedance magnitude is within the range defined by the upper and lower predetermined thresholds (i.e., a value simultaneously above the lower predetermined threshold and below the upper predetermined threshold), system may maintain (2712) the current algorithm for the RF therapeutic energy activation cycle. However if system (98) determines the newly calculated mean average impedance magnitude value is outside the determined range, system (98) may alter (2714) RF algorithm used for the RF therapeutic energy activation cycle in order to compensate for the increase/decrease in the mean average impedance value.

The upper predetermined threshold and the lower predetermined threshold may be a percentage increase/decrease from the baseline value. Alternatively, the upper predetermined threshold and the lower predetermined threshold may be a predetermined value above and below the baseline value. The difference between the upper predetermined threshold and the starting value may be different than the difference between the lower predetermined threshold and the starting value.

While in the current example, the previous five RF therapeutic energy activation cycles are utilized to calculate the mean average impedance magnitude value, any suitable number of RF energy activation cycles may be used as would be apparent to one skilled in the art in view of the teachings herein. For example, the previous 2 RF therapeutic energy activation cycles may be used, the previous 10 RF therapeutic energy activation cycles may be used, the previous 50 RF therapeutic energy activation cycles may be used, etc.

### IV. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument comprising: (a) an end effector including: (i) a first jaw, (ii) a second jaw, wherein the first jaw and the second jaw are configured to move relative to each other between an open position and a clamped position in order to grasp tissue between the first jaw and the second jaw in the clamped position, and (iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue; and (b) a controller configured to measure and analyze an impedance parameter associated with the end effector, wherein the controller is configured to compare the measured impedance parameter with an upper predetermined threshold, wherein the controller is configured to generate a cleaning instruction or alter an energy delivery algorithm of the RF therapeutic energy cycle if the measured impedance parameter is greater than the upper predetermined threshold.

### Example 2

The surgical instrument of Example 1, wherein the end effector is configured to emit non-therapeutic energy, wherein the impedance parameter comprises an impedance magnitude in response to the emitted non-therapeutic energy.

### Example 3

The surgical instrument of Example 2, wherein the controller is configured to only measure the impedance magnitude of the impedance parameter while the first jaw and the second jaw are in the open position.

### Example 4

The surgical instrument of Example 3, wherein the electrode is configured to emit non-therapeutic energy.

### Example 5

The surgical instrument of any one or more of Examples 1-4, wherein the impedance parameter comprises an impedance magnitude in response to the electrode emitting the RF therapeutic energy cycle.

### Example 6

The surgical instrument of Example 5, wherein the impedance magnitude comprises a minimum impedance magnitude of the RF therapeutic energy cycle.

### Example 7

The surgical instrument of any one of Examples 1 through 6, wherein the impedance parameter comprises a mean impedance magnitude calculated from a plurality of RF therapeutic energy cycles.

### Example 8

The surgical instrument of Example 7, wherein the mean impedance magnitude is calculated from an average impedance magnitude of each RF therapeutic energy cycle of the plurality of RF therapeutic energy cycles.

### Example 9

The surgical instrument of any one or more of Examples 1 through 8, wherein the controller is configured to generate a cleaning instruction or alter an energy delivery algorithm of the RF therapeutic energy cycle if the measured impedance parameter is lower than a lower predetermined threshold.

### Example 10

The surgical instrument of any one or more of Examples 1 through 10, further comprising a second electrode associated with the second jaw, wherein the first electrode and the second electrode are configured to emit bipolar RF therapeutic energy.

### Example 11

A surgical instrument comprising: (a) an end effector including: (i) a first jaw, (ii) a second jaw, wherein the first jaw and the second jaw are configured to move relative to each other between an open position and a clamped position in order to grasp tissue between the first jaw and the second jaw in the clamped position, and (iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue; (b) a sensor configured to measure a jaw opening rate of either the first jaw or the second jaw as the first jaw and the second jaw move from the clamped position to the open position; and (c) a controller in communication with the sensor, wherein the controller is configured to compare the measured jaw opening rate with a predetermined threshold, wherein the controller is configured to generate a cleaning instruction or alter an energy delivery algorithm of the RF therapeutic energy cycle if the measured jaw opening rate is below the predetermined threshold.

### Example 12

The surgical instrument of Example 11, further comprising a shaft assembly extending proximally from the electrode, wherein the sensor is associated with the shaft assembly.

### Example 13

The surgical instrument of any one or more of Examples 11 through 12, wherein the first jaw and the second jaw are pivotally coupled to each other, wherein the sensor comprises an inclinometer.

### Example 14

The surgical instrument of any one or more of Examples 11 through 13, wherein the end effector comprises a second electrode associated with the second jaw.

### Example 15

The surgical instrument of any one or more of Examples 11 through 15, further comprising a handle, wherein the controller is located within the handle.

### Example 16

A surgical instrument comprising: (a) an end effector including: (i) a first jaw, (ii) a second jaw, wherein the first jaw and the second jaw are configured to move relative to each other between an open position and a clamped position in order to grasp tissue between the first jaw and the second jaw in the clamped position, and (iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue; (b) a sensor configured to measure a jaw gap between the first jaw and the second jaw in the clamped position; and (c) a controller in communication with the sensor, wherein the controller is configured to compare the measured jaw gap with a predetermined threshold, wherein the controller is configured to generate a cleaning instruction or alter an energy delivery algorithm of the RF therapeutic energy cycle if the measured jaw gap is above the predetermined threshold.

### Example 17

The surgical instrument of Example 16, further comprising a waveform generator configured to activate the electrode with RF therapeutic energy.

### Example 18

The surgical instrument of Example 17, wherein the controller is located within the waveform generator.

### Example 19

The surgical instrument of any one or more of Examples 16 through 18, further comprising a clamp sensor in commutation with the controller, wherein the clamp sensor is configured to detect when the first jaw and the second jaw are in the clamped position.

### Example 20

The surgical instrument of Example 19, wherein the clamp sensor is in communication with the controller.

### V. Miscellaneous

Any one or more of the teaching, expressions, embodiments, examples, etc. described herein may be combined with any one or more of the teachings, expressions, embodiments, examples, etc. described in U.S. Pat. App. No. [Atty. Ref. No. END9573USNP1], entitled “Electrosurgical Instrument with Impedance Spectroscopy and Method of Monitoring State of Instrument Jaws and Tissue,” filed on even date herewith; U.S. Pat. App. No. [Atty. Ref. No. END9574USNP1], entitled “Electrosurgical Instrument with Jaw Status Monitoring and Method of Adjusting Energy Activation,” filed on even date herewith; U.S. Pat. App. No. [Atty. Ref. No. END9575USNP1], entitled “Electrosurgical Instrument and Method of Monitoring Clamp Position to Adjust Energy Application,” filed on even date herewith; U.S. Pat. App. No. [Atty. Ref. No. END9577USNP1], entitled “Electrosurgical Instrument and Method of Applying Energy,” filed on even date herewith; and/or U.S. Pat. App. No. [Atty. Ref. No. END9609USNP1], entitled “Electrosurgical Instrument and Method of Frequency Monitoring for Sealing Tissue,” filed on even date herewith. The disclosure of each of these applications is incorporated by reference herein.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument comprising:
(a) an end effector including:
(i) a first jaw,
(ii) a second jaw, wherein the first jaw and the second jaw are configured to move relative to each other between an open position and a clamped position in order to grasp tissue between the first jaw and the second jaw in the clamped position, and
(iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue; and
(b) a controller configured to measure and analyze an impedance parameter associated with the end effector, wherein the controller is configured to compare the measured impedance parameter with an upper predetermined threshold, wherein the controller is configured to generate a cleaning instruction or alter an energy delivery algorithm of the RF therapeutic energy cycle if the measured impedance parameter is greater than the upper predetermined threshold.

2. The surgical instrument of claim 1, wherein the end effector is configured to emit non-therapeutic energy, wherein the impedance parameter comprises an impedance magnitude in response to the emitted non-therapeutic energy.

3. The surgical instrument of claim 2, wherein the controller is configured to only measure the impedance magnitude of the impedance parameter while the first jaw and the second jaw are in the open position.

4. The surgical instrument of claim 3, wherein the electrode is configured to emit non-therapeutic energy.

5. The surgical instrument of claim 1, wherein the impedance parameter comprises an impedance magnitude in response to the electrode emitting the RF therapeutic energy cycle.

6. The surgical instrument of claim 5, wherein the impedance magnitude comprises a minimum impedance magnitude of the RF therapeutic energy cycle.

7. The surgical instrument of claim 1, wherein the impedance parameter comprises a mean impedance magnitude calculated from a plurality of RF therapeutic energy cycles.

8. The surgical instrument of claim 7, wherein the mean impedance magnitude is calculated from an average impedance magnitude of each RF therapeutic energy cycle of the plurality of RF therapeutic energy cycles.

9. The surgical instrument of claim 1, wherein the controller is configured to generate a cleaning instruction or alter an energy delivery algorithm of the RF therapeutic energy cycle if the measured impedance parameter is lower than a lower predetermined threshold.

10. The surgical instrument of claim 1, further comprising a second electrode associated with the second jaw, wherein the first electrode and the second electrode are configured to emit bipolar RF therapeutic energy.

11. A surgical instrument comprising:
(a) an end effector including:
(i) a first jaw,
(ii) a second jaw, wherein the first jaw and the second jaw are configured to move relative to each other between an open position and a clamped position in order to grasp tissue between the first jaw and the second jaw in the clamped position, and
(iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue;
(b) a sensor configured to measure a jaw opening rate of either the first jaw or the second jaw as the first jaw and the second jaw move from the clamped position to the open position; and
(c) a controller in communication with the sensor, wherein the controller is configured to compare the measured jaw opening rate with a predetermined threshold, wherein the controller is configured to generate a cleaning instruction or alter an energy delivery algorithm of the RF therapeutic energy cycle if the measured jaw opening rate is below the predetermined threshold.

12. The surgical instrument of claim 11, further comprising a shaft assembly extending proximally from the electrode, wherein the sensor is associated with the shaft assembly.

13. The surgical instrument of claim 11, wherein the first jaw and the second jaw are pivotally coupled to each other, wherein the sensor comprises an inclinometer.

14. The surgical instrument of claim 11, wherein the end effector comprises a second electrode associated with the second jaw.

15. The surgical instrument of claim 11, further comprising a handle, wherein the controller is located within the handle.

16. A surgical instrument comprising:
(a) an end effector including:
(i) a first jaw,
(ii) a second jaw, wherein the first jaw and the second jaw are configured to move relative to each other between an open position and a clamped position in order to grasp tissue between the first jaw and the second jaw in the clamped position, and
(iii) an electrode associated with the first jaw, wherein the electrode is configured to engage the grasped tissue while the first jaw and the second jaw are in the clamped position, wherein the electrode is configured to emit an RF therapeutic energy cycle to the grasped tissue in the clamped position to thereby seal the grasped tissue;
(b) a sensor configured to measure a jaw gap between the first jaw and the second jaw in the clamped position; and
(c) a controller in communication with the sensor, wherein the controller is configured to compare the measured jaw gap with a predetermined threshold, wherein the controller is configured to generate a cleaning instruction or alter an energy delivery algorithm of the RF therapeutic energy cycle if the measured jaw gap is above the predetermined threshold.

17. The surgical instrument of claim 16, further comprising a waveform generator configured to activate the electrode with RF therapeutic energy.

18. The surgical instrument of claim 17, wherein the controller is located within the waveform generator.

19. The surgical instrument of claim 16, further comprising a clamp sensor in commutation with the controller, wherein the clamp sensor is configured to detect when the first jaw and the second jaw are in the clamped position.

20. The surgical instrument of claim 19, wherein the clamp sensor is in communication with the controller.
